(19) United States
(12) Patent Application Publication
(10) Pub. No.: US 2022/0309669 A1
(43) Pub. Date: Sep. 29, 2022

(54) MEDICAL IMAGE DIAGNOSIS SYSTEM AND NON-TRANSITORY COMPUTER-READABLE STORAGE MEDIUM STORING THEREIN IMAGE PROCESSING PROGRAM

(71) Applicant: CANON MEDICAL SYSTEMS CORPORATION, Otawara-shi (JP)

(72) Inventor: Yutaka HOSHIYAMA, Nasushiobara (JP)

(73) Assignee: CANON MEDICAL SYSTEMS CORPORATION, Otawara-shi (JP)

(21) Appl. No.: 17/649,855

(22) Filed: Feb. 3, 2022

(30) Foreign Application Priority Data
Mar. 25, 2021 (JP) 2021-051860

(51) Int. Cl. G06T 7/00; G06T 7/13; G16H 30/40

(52) U.S. Cl. CPC G06T 7/0014; G06T 7/13; G16H 30/40; G06T 2207/30081; G06T 2207/10088; G06T 2207/30096

(57) ABSTRACT

A medical image diagnosis system according to an embodiment includes processing circuitry. The processing circuitry is configured to detect a first area corresponding to an organ of an examined subject from a first medical image. The processing circuitry is configured to detect a second area corresponding to a tumor in the organ from one of the first medical image and a second medical image different from the first medical image. The processing circuitry is configured to specify a boundary of the organ on the basis of the first area and the second area. The processing circuitry is configured to determine a slice plane by using the boundary as a reference.

### CROSS-REFERENCE TO RELATED APPLICATIONS

This application is based upon and claims the benefit of priority from Japanese Patent Application No. 2021-051860, filed on Mar. 25, 2021; the entire contents of which are incorporated herein by reference.

### FIELD

Embodiments described herein relate generally to a magnetic resonance imaging apparatus, a medical image diagnosis system, and a non-transitory computer-readable storage medium storing therein an image processing program.

### BACKGROUND

Conventionally, in Magnetic Resonance Imaging (MRI) examinations on the prostate, extracapsular extension of a prostate tumor may be evaluated. In the evaluation of the extracapsular extension, it is difficult, particularly in the prostate apex, to diagnose extracapsular extension from a simple transverse cross-sectional image (an axial image) due to partial volume effect. For this reason, in order to diagnose extracapsular extension, an assessment may be made from observations in multiple directions using not only the transverse cross-sectional image but also a coronal cross-sectional image (a coronal image) and a sagittal cross-sectional image (a sagittal image), or an assessment may be made from an observation using a Multi Planar Reconstruction [MPR] (cross-section transformation) image generated after three-dimensional (3D) imaging. However, the observations using these images may be cumbersome for users in some situations.

Further, setting a slice plane by using a medical image is known. However, it is difficult to set a perpendicular slice plane at the boundary between a prostate tumor and the prostate.

### BRIEF DESCRIPTION OF THE DRAWINGS

FIG. 1 is a block diagram illustrating an example of a medical image diagnosis system according to an embodiment;

FIG. 2 is a block diagram illustrating an example of a magnetic resonance imaging apparatus according to the embodiment;

FIG. 3 is a flowchart illustrating an example of a procedure in a slice determining process according to the embodiment;

FIG. 4 is a drawing according to the embodiment illustrating examples of a coronal image related to the prostate of an examined subject and a prostate image including a prostate region extracted from the coronal image;

FIG. 5 is a drawing according to the embodiment illustrating other examples of a coronal image related to the prostate of the examined subject and a prostate image including a prostate region extracted from the coronal image;

FIG. 6 is a drawing according to the embodiment illustrating examples of a coronal image related to the prostate of the examined subject and a tumor image including a tumor extracted from the coronal image;

FIG. 7 is a drawing according to the embodiment illustrating other examples of a coronal image related to the prostate of the examined subject and a tumor image including a tumor extracted from the coronal image;

FIG. 8 is a drawing according to the embodiment illustrating examples of a target boundary area specified on the basis of the prostate region in FIG. 4 and a tumor region in the FIG. 5 and an approximation straight line;

FIG. 9 is a drawing according to the embodiment illustrating examples of a target boundary area specified on the basis of a prostate region in FIG. 6 and a tumor region in FIG. 7 and an approximation straight line;

FIG. 10 is a drawing according to the embodiment illustrating examples of a reference plane determined on the basis of the target boundary area and the approximation straight line in FIG. 8 and a group of slices including the reference plane and a plurality of determined planes;

FIG. 11 is a drawing according to the embodiment illustrating examples of a reference plane determined on the basis of the target boundary area and the approximation straight line in FIG. 9 and a group of slices including the reference plane and a plurality of determined planes;

FIG. 12 is a drawing according to the embodiment illustrating an example of a superimposition image displayed by superimposing a tumor region in FIG. 6 and the group of slices in FIG. 10 on the prostate region in FIG. 4; and

FIG. 13 is a drawing according to the embodiment illustrating a superimposition image displayed by superimposing the tumor region in FIG. 7 and the group of slices in FIG. 11 on the prostate region in FIG. 5.

### DETAILED DESCRIPTION

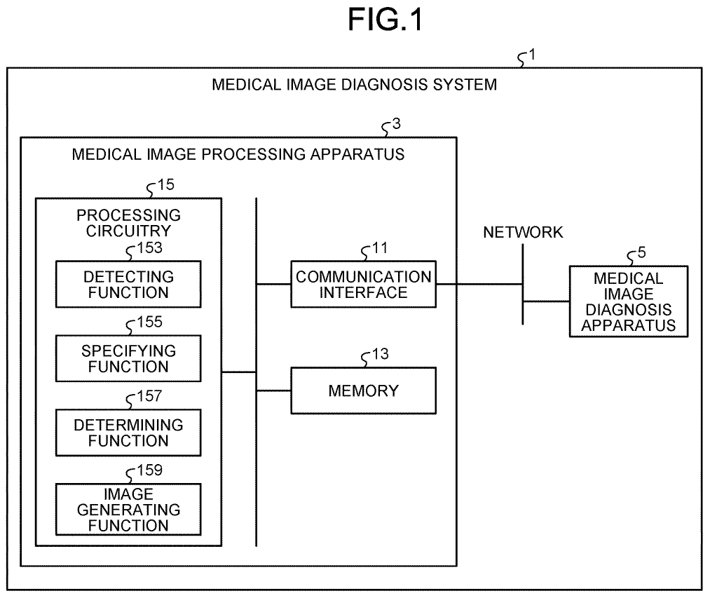

Exemplary embodiments of a medical image diagnosis system and a medical image processing program will be explained in detail below, with reference to the accompanying drawings. FIG. 1 is a block diagram illustrating an example of a medical image diagnosis system 1 according to an embodiment. The medical image diagnosis system 1 includes a medical image processing apparatus 3 and a medical image diagnosis apparatus 5. The medical image processing apparatus 3 and the medical image diagnosis apparatus 5 are connected via a network. In this situation, to the network, a server of a medical image management system (hereinafter, “Picture Archiving and Communication Systems [PACS]”), a server of a hospital information system (hereinafter, “HIS”), and or the like may be connected. Further, various types of functions of the medical image processing apparatus 3 may be installed in any of various types of servers and/or the medical image diagnosis apparatus 5. The medical image diagnosis apparatus 5 may be any of various types of modalities, for example.

Examples of the modalities include Magnetic Resonance Imaging (hereinafter, “MRI”) apparatuses, X-ray computed tomography apparatuses, and ultrasound diagnosis apparatuses. For example, when the medical image diagnosis apparatus 5 is an MRI apparatus, the medical image diagnosis system 1 corresponds to a magnetic resonance imaging system. In the following sections, to explain specific example, it is assumed that the various types of functions of the medical image processing apparatus 3 are installed in the MRI apparatus. In this situation, the MRI apparatus includes various types of functions of processing circuitry 15.

The medical image diagnosis system 1 according to the embodiment includes the processing circuitry 15. The processing circuitry 15 is configured to detect a first area corresponding to an organ of an examined subject (hereinafter, “patient”) from a first medical image. The processing circuitry is configured to detect a second area corresponding to a tumor in the organ from one of the first medical image and a second medical image different from the first medical image. On the basis of the first area and the second area, the processing circuitry is configured to specify the boundary of the organ. The processing circuitry is configured to determine a slice plane by using the boundary as a reference.

### Embodiments

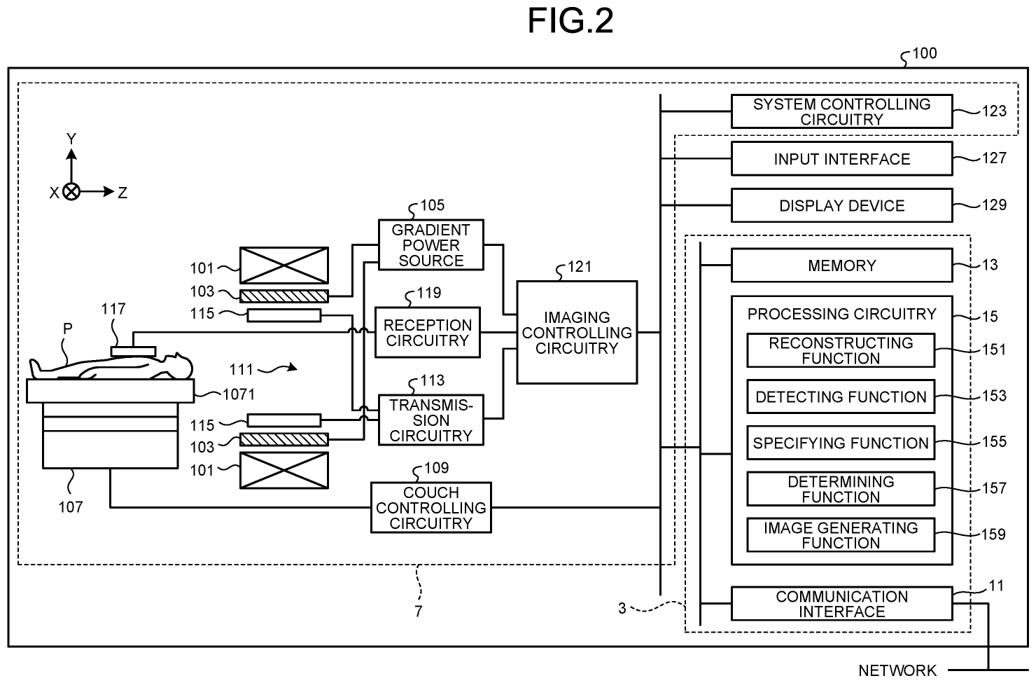

FIG. 2 is a diagram illustrating an example of an MRI apparatus 100 according to the present embodiment. As illustrated in FIG. 2, the MRI apparatus 100 includes an imaging unit (an imaging apparatus) 7, an input interface 127, a display device 129, and a medical image processing apparatus 3. The imaging unit 7 has a function of imaging a patient P. The imaging unit 7 includes a static magnetic field magnet 101, a gradient coil 103, a gradient power source 105, a couch 107, couch controlling circuitry 109, transmission circuitry 113, a transmission coil 115, a reception coil 117, reception circuitry 119, imaging controlling circuitry (an imaging controlling unit) 121, and system controlling circuitry (a system controlling unit) 123. The medical image processing apparatus 3 may further include the input interface 127 and the display device 129, in addition to a communication interface 11, a memory 13, and the processing circuitry 15.

The static magnetic field magnet 101 is a magnet formed so as to have a hollow and substantially circular cylindrical shape. The static magnetic field magnet 101 is configured to generate a substantially uniform static magnetic field in a space on the inside thereof. For example, a superconductive magnet or the like may be used as the static magnetic field magnet 101.

The gradient coil 103 is a coil formed to have a hollow and substantially circular cylindrical shape and is arranged on the inner surface side of a cooling container having a circular cylindrical shape. By individually receiving a supply of an electric current from the gradient power source 105, the gradient coil 103 is configured to generate gradient magnetic fields of which the magnetic field intensities change along X-, Y-, and Z- axes that are orthogonal to one another. For example, the gradient magnetic fields generated by the gradient coil 103 along the X-, Y-, and Z- axes form a slice selecting-purpose gradient magnetic field, a phase encoding-purpose gradient magnetic field, and a frequency encoding-purpose gradient magnetic field. The slice selecting-purpose gradient magnetic field is used for arbitrarily determining an imaged cross-sectional plane. The phase encoding-purpose gradient magnetic field is used for changing the phase of a magnetic resonance signal (hereinafter, “MR signal”) in accordance with a spatial position. The frequency encoding-purpose gradient magnetic field is used for changing the frequency of an MR signal in accordance with a spatial position.

The gradient power source 105 is a power source apparatus configured to supply the electric currents to the gradient coil 103, under control of the imaging controlling circuitry 121.

The couch 107 is an apparatus including a couchtop 1071 on which the patient P is placed. Under control of the couch controlling circuitry 109, the couch 107 is configured to insert the couchtop 1071 on which the patient P is placed, into a bore 111.

The couch controlling circuitry 109 is circuitry configured to control the couch 107. By driving the couch 107 according to an instruction from an operator received via an input/output interface 17, the couch controlling circuitry 109 moves the couchtop 1071 in longitudinal directions and up-and-down directions, as well as left-and-right directions in some situations.

The transmission circuitry 113 is configured to supply a radio frequency pulse modulated with a Larmor frequency to the transmission coil 115, under control of the imaging controlling circuitry 121. For example, the transmission circuitry 113 includes an oscillating unit, a phase selecting unit, a frequency converting unit, an amplitude modulating unit, a Radio Frequency (RF) amplifier, and the like. The oscillating unit is configured to generate an RF pulse having a resonance frequency unique to a target atomic nucleus positioned in the static magnetic field. The phase selecting unit is configured to select a phase of the RF pulse generated by the oscillating unit. The frequency converting unit is configured to convert the frequency of the RF pulse output from the phase selecting unit. The amplitude modulating unit is configured to modulate the amplitude of the RF pulse output from the frequency converting unit according to a sinc mathematical function, for example. The RF amplifier is configured to amplify the RF pulse output from the amplitude modulating unit and to supply the amplified RF pulse to the transmission coil 115.

The transmission coil 115 is a Radio Frequency (RF) coil arranged on the inside of the gradient coil 103. The transmission coil 115 is configured to generate an RF pulse corresponding to a radio frequency magnetic field, in accordance with the output from the transmission circuitry 113.

The reception coil 117 is an RF coil arranged on the inside of the gradient coil 103. The reception coil 117 is configured to receive an MR signal emitted from the patient P due to the radio frequency magnetic field. The reception coil 117 is configured to output the received MR signal to the reception circuitry 119. For example, the reception coil 117 is a coil array including one or more, typically two or more, coil elements. In the following sections, to explain a specific example, the reception coil 117 is assumed to be a coil array including two or more coil elements.

Alternatively, the reception coil 117 may be structured with one coil element. Further, although FIG. 2 depicts the transmission coil 115 and the reception coil 117 as separate RF coils, the transmission coil 115 and the reception coil 117 may be implemented as an integrally-formed transmission/reception coil. The transmission/reception coil corresponds to an imaged site of the patient P and may be, for example, a local transmission/reception RF coil such as a head coil.

On the basis of the MR signal output from the reception coil 117, the reception circuitry 119 is configured to generate a digital MR signal (hereinafter, “MR data”) under control of the imaging controlling circuitry 121. More specifically, the reception circuitry 119 generates the MR data by performing signal processing processes such as detecting and filtering processes on the MR signal output from the reception coil 117 and subsequently performing an Analog-to-Digital conversion (hereinafter, “A/D conversion”) on the data resulting from the signal processing processes. The reception circuitry 119 is configured to output the generated MR data to the imaging controlling circuitry 121. For example, the MR data is generated for each of the coil elements and is output to the imaging controlling circuitry 121 together with a tag identifying the relevant coil element.

The imaging controlling circuitry 121 is configured to perform an imaging process on the patient P, by controlling the gradient power source 105, the transmission circuitry 113, the reception circuitry 119, and the like according to an imaging protocol output from the processing circuitry 15. The imaging protocol includes a pulse sequence corresponding to the type of the medical examination. For example, the imaging protocol defines: the magnitude of the electric current to be supplied to the gradient coil 103 by the gradient power source 105; the timing with which the electric current is to be supplied to the gradient coil 103 by the gradient power source 105; the magnitude and the time width of the radio frequency pulse to be supplied to the transmission coil 115 by the transmission circuitry 113; the timing with which the radio frequency pulse is to be supplied to the transmission coil 115 by the transmission circuitry 113; the timing with which the MR signal is to be received by the reception coil 117; and the like. When having received the MR data from the reception circuitry 119 as a result of imaging the patient P by driving the gradient power source 105, the transmission circuitry 113, the reception circuitry 119, and the like, the imaging controlling circuitry 121 is configured to store the received MR data into the memory 13. The imaging controlling circuitry 121 is realized by using a processor, for example.

The term “processor” denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]).

As a hardware resource, the system controlling circuitry 123 includes a processor, memory elements such as a Read-Only Memory (ROM), a Random Access Memory (RAM), and/or the like (not illustrated) and is configured to control the MRI apparatus 100 by employing a system controlling function. More specifically, the system controlling circuitry 123 reads a system controlling program stored in a memory, loads the read program into a memory, and controls pieces of circuitry of the MRI apparatus 100 according to the loaded system controlling program.

For example, on the basis of imaging conditions input by the operator via the input interface 127, the system controlling circuitry 123 is configured to read an imaging protocol from the memory 13. The system controlling circuitry 123 is configured to transmit the imaging protocol to the imaging controlling circuitry 121 so as to control the imaging process performed on the patient P. The system controlling circuitry 123 is realized by using a processor, for example. Alternatively, the system controlling circuitry 123 may be incorporated in the processing circuitry 15. In that situation, the system controlling function is executed by the processing circuitry 15, so that the processing circuitry 15 functions as a substitute for the system controlling circuitry 123. Because the processor to realize the system controlling circuitry 123 is the same as described above, the explanations thereof will be omitted.

The memory 13 is configured to store therein various types of programs related to the system controlling function executed by the system controlling circuitry 123, various types of imaging protocols, imaging conditions including a plurality of imaging parameters defining the imaging protocols, and the like. Further, the memory 13 is configured to store therein a reconstructing function 151, a detecting function 153, a specifying function 155, a determining function 157, and an image generating function 159 realized by the processing circuitry 15, in the form of computer-executable programs. Further, the memory 13 stores therein a pre-scan image generated through a pre-scan such as a locator scan and a medical image generated through a main scan performed after the pre-scan.

The memory 13 is configured to store therein MR data related to the main scan and an algorithm used for reconstructing an MR image on the basis of the MR data. Further, the memory 13 may store therein various types of data received via the communication interface 11. For example, the memory 13 stores therein information (an imaged site, a medical examination purpose, etc.) related to a medical examination order for the patient P received from an information processing system in the medical institution such as a Radiology Information System (RIS) or the like.

For example, the memory 13 is realized by using a semiconductor memory element such as a ROM, a RAM, or a flash memory, or a Hard Disk Drive (HDD), a Solid State Drive (SSD), an optical disk, or the like. Alternatively, the memory 13 may be realized by using a Compact Disc (CD)-ROM drive, a Digital Versatile Disc (DVD) drive, or a drive apparatus or the like that reads and writes various types of information from and to a portable storage medium such as a flash memory.

The input interface 127 is configured to receive various types of instructions and inputs of information from the operator. For example, the input interface 127 is realized by using a trackball, a switch button, a mouse, a keyboard, a touchpad on which input operations can be performed by touching an operation surface thereof, a touch screen in which a display screen and a touchpad are integrally formed, contactless input circuitry using an optical sensor, audio input circuitry, and/or the like. The input interface 127 is connected to the processing circuitry 15 and is configured to convert input operations received from the operator into electrical signals and to output the electrical signals to the processing circuitry 15. In the present disclosure, the input interface 127 does not necessarily have to include physical operation component parts such as the mouse, the keyboard, and/or the like. Possible examples of the input interface 127 include electrical signal processing circuitry configured to receive an electrical signal corresponding to an input operation from an external input device provided separately from the MRI apparatus 100 and to output the electrical signal to controlling circuitry.

Under control of either the processing circuitry 15 or the system controlling circuitry 123, the display device 129 is configured to display various types of Graphical User Interfaces (GUIs), MR images generated by the processing circuitry 15, and the like. For example, the display device 129 is realized by using a Cathode Ray Tube (CRT) display device, a liquid crystal display device, an organic Electroluminescence (EL) display device, a Light Emitting Diode (LED) display device, a plasma display device, or any other arbitrary display device such as a display apparatus or a monitor known in the relevant technical field. The display device 129 corresponds to a display unit.

The medical image processing apparatus 3 includes the communication interface 11, the memory 13, and the processing circuitry 15.

The communication interface 11 is configured to carry out data communication with the HIS, the PACS, and the like, for example. The communication between the communication interface 11 and the hospital information systems may use any standard, but possible examples of the standard include Health Level 7 (HL7), Digital Imaging and Communication in Medicine (DICOM), and using both of these two. The communication interface 11 is configured to receive the information (the imaged site, the medical examination purpose, etc.) related to the medical examination order for the patient P received from the information processing system in the medical institution such as the Radiology Information System (RIS) or the like. Further, when the medical image processing apparatus 3 is not installed in the MRI apparatus 100, the communication interface 11 included in the medical image processing apparatus 3 is configured to receive MR images from, for example, the MRI apparatus 100 that images the patient P in the medical examination performed on the patient P. In that situation, the received MR images are stored in the memory 13.

For example, the processing circuitry 15 is realized by using the processor described above or the like. The processing circuitry 15 includes, among others, the reconstructing function 151, the detecting function 153, the specifying function 155, the determining function 157, and the image generating function 159. The processing circuitry 15 that realizes the reconstructing function 151, the detecting function 153, the specifying function 155, the determining function 157, and the image generating function 159 corresponds to a reconstructing unit, a detecting unit, a specifying unit, a determining unit, and an image generating unit, respectively. The functions such as the reconstructing function 151, the detecting function 153, the specifying function 155, the determining function 157, and the image generating function 159 are stored in the memory 13 in the form of computer-executable programs. For example, the processing circuitry 15 realizes the functions corresponding to the programs, by reading and executing the programs from the memory 13. In other words, the processing circuitry 15 that has read the programs has the functions such as the reconstructing function 151, the detecting function 153, the specifying function 155, the determining function 157, and the image generating function 159.

In the above description, the example was explained in which the “processor” reads and executes the programs corresponding to the functions from the memory 13; however, possible embodiments are not limited to this example. For instance, when the processor is a CPU, the processor realizes the functions by reading and executing the programs saved in the memory 13. In contrast, when the processor is an ASIC, the functions are directly incorporated in the circuitry of the processor as logic circuitry, instead of the programs being saved in the memory 13. Further, the processors according to the present embodiment do not each necessarily have to be structured as a single piece of circuitry. It is also acceptable to structure one processor by combining together a plurality of pieces of independent circuitry so as to realize the functions thereof. Further, although the example was explained in which the single piece of storage circuitry stores therein the programs corresponding to the processing functions, it is also acceptable to arrange a plurality of pieces of storage circuitry in a distributed manner, so that the processing circuitry 15 reads a corresponding program from each of the individual pieces of storage circuitry.

By employing the reconstructing function 151, the processing circuitry 15 is configured to obtain, from the reception circuitry 119, the MR data generated through the scan performed on the patient P, to arrange the obtained MR data in a k-space, and to reconstruct medical images on the basis of the MR data arranged in the k-space. As for the reconstruction of the medical images, because it is possible to use an existing reconstruction method, explanations thereof will be omitted.

By employing the detecting function 153, the processing circuitry 15 is configured to detect a first area corresponding to an organ of the patient P from a first medical image reconstructed by the reconstructing function 151. The detecting function 153 is configured to detect a second area corresponding to a tumor in the organ from one of the first medical image and a second medical image different from the first medical image. Further, although the example is explained above in which the first area and the second area are detected from the one or more medical images, the first area and the second area may be detected from volume data rendering the organ and the tumor. In the following sections, to explain a specific example, it is assumed that the organ is the prostate, while the tumor is prostate cancer, and the first area and the second area are detected from one or more coronal images.

The first area corresponds to an area representing the prostate in the first medical image. The second area corresponds to an area representing the prostate cancer in one of the first medical image and the second medical image. The first medical image is of an image type suitable for the detection of the area representing the prostate or the detection of the areas representing the prostate and the prostate cancer. Further, when the first medical image is of the image type suitable for the detection of the area representing the prostate, the second medical image is of an image type that is suitable for the detection of the area representing the prostate cancer and is different from the image type of the first medical image, and may have contrast different from that of the first medical image, for example. In this situation, during the main scan performed after the pre-scan, for example, a scan is carried out to acquire MR data related to the first medical image, and subsequently, another scan is carried out to acquire MR data related to the second medical image.

For example, by performing a segmentation process (e.g., a region extracting process) on the first medical image, the detecting function 153 is configured to extract, as the first area, a region representing the prostate (hereinafter, “prostate region”) from the first medical image. In an example, by performing a contour extracting process on the first medical image, the detecting function 153 may extract, as the first area, the contour of the prostate region from the first medical image. Further, by performing a segmentation process on one of the first medical image and the second medical image, the detecting function 153 is configured to extract, as the second area, a region representing the prostate cancer (hereinafter, “tumor region”) from the medical image to which the segmentation process has been applied. In an example, by performing a contour extracting process on one of the first medical image and the second medical image, the detecting function 153 may extract, as the second area, the contour of the tumor region from the medical image to which the contour extracting process has been applied.

For the segmentation process and the contour extracting process, it is possible to use an existing process, as appropriate. For example, the segmentation process and the contour extracting process may be realized by threshold value processing using pixel values, a rule-based algorithm for region extraction or contour extraction, or a trained Deep Neural Network (DNN) (a trained model). A processing program or the trained model related to the segmentation process and the contour extracting process is stored in the memory 13. In an example, the segmentation process and the contour extracting process may be realized by a multi segmentation process and a multi contour extracting process in which, when one medical image is input, the first area and the second area are output. In addition to the first area and the second area, the multi segmentation process and the multi contour extracting process may extract an area that is neither the first area nor the second area. Specific examples related to the detecting function 153 will be explained later.

By employing the specifying function 155, the processing circuitry 15 is configured to specify the boundary of the organ on the basis of the first area and the second area. More specifically, the specifying function 155 is configured to specify the boundary of the organ related to the tumor, on the basis of the position of the second area relative to the first area. Even more specifically, for example, the specifying function 155 is configured to specify, as the boundary, a boundary line (hereinafter, “target boundary area”) between a part of the second area that overlaps with the first area and a part of the second area positioned outside the first area. For example, the target boundary area corresponds to the boundary line between a part of the tumor region of the prostate cancer that overlaps with the prostate region and a part of the tumor region positioned outside the prostate region. Alternatively, the specifying function 155 may specify, as the boundary, a part of the contour of the first area that is distant from the second area by a distance equal to or shorter than a predetermined length. In other words, even when the tumor region is included in the prostate region, while the tumor region is not in contact with the contour of the prostate region, for example, the specifying function 155 is able to specify the boundary of the prostate region. In this situation, the predetermined length may be zero. In other words, the specifying function 155 may specify, as the boundary of the organ related to the tumor, a part of the contour of the first area that overlaps with or is in contact with the second area. When the data used by the detecting function 153 is volume data, the target boundary area corresponds to a boundary plane between the part of the second area that overlaps with the first area and the part of the second area positioned outside the first area. Specific examples of the specifying function 155 will be explained later.

By employing the determining function 157, the processing circuitry 15 is configured to determine a slice plane, by using the boundary as a reference. The determined slice plane is related to a diagnosis process on the tumor, for example. More specifically, the determining function 157 is configured to determine the slice plane so as to intersect the boundary. Even more specifically, the determining function 157 is configured to determine the slice plane so as to perpendicularly intersect the boundary. For example, the determining function 157 determines, as the slice plane, a plane that includes a perpendicular bisector of the line segment connecting together the two end points of the boundary and that is perpendicular to the medical image used by the detecting function 153. In this situation, while using the determined slice plane as a reference (hereinafter, “reference plane”), the determining function 157 determines a plurality of planes that are parallel to the reference plane, up to each of the two end points. Alternatively, the determining function 157 may determine a plurality of planes that are parallel to the reference plane, from the reference plane to an end part of an imaged region.

Further, when the boundary is a curve, the determining function 157 may determine, as the reference plane, a plane that includes a perpendicular bisector of the longest line segment at the boundary and that is perpendicular to the medical image used by the detecting function 153. Further, when the boundary is substantially semicircular, the determining function 157 may determine, as the reference plane, a plane that includes a line perpendicular to the tangent line at the middle point of the boundary and that is perpendicular to the medical image used by the detecting function 153. Specific examples related to the determining function 157 will be explained later.

By employing the image generating function 159, the processing circuitry 15 is configured to perform various types of image processing processes on the medical images, the volume data, and the like generated by the reconstructing function 151, so as to generate medical images on which the image processing processes have been performed. Examples of the image processing processes include a contrast adjusting process, a cross-section transformation (Multi-Planar Reconstruction) process, and rendering process. The image generating function 159 is configured to store the medical images generated through the various types of image processing processes into the memory 13.

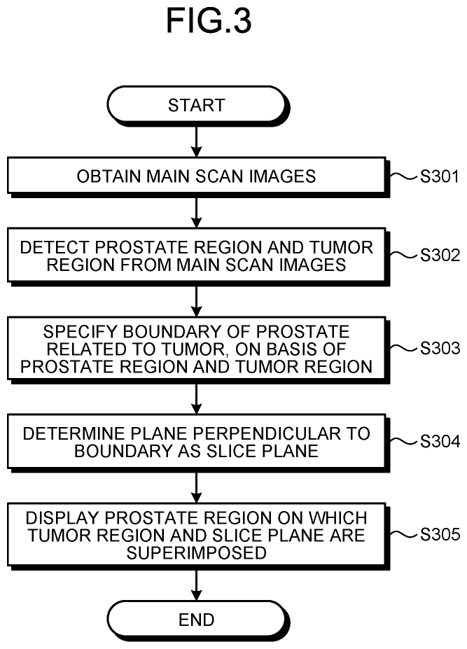

The process to determine the slice plane (hereinafter, “slice determining process”) performed by the MRI apparatus 100 or the medical image processing apparatus 3 according to the present embodiment configured as described above will be explained with reference to FIGS. 3 to 13. FIG. 3 is a flowchart illustrating an example of a procedure in the slice determining process. In the following sections, to make the explanation simpler, the first medical image and the second medical image will collectively be referred to as main scan images.

### The Slice Determining Process:

Step S301:

The processing circuitry 15 obtains, from the memory 13, the main scan images generated through the main scan performed on the patient P. Alternatively, in place of the main scan images, pre-scan images may be used in the following processes.

Step S302:

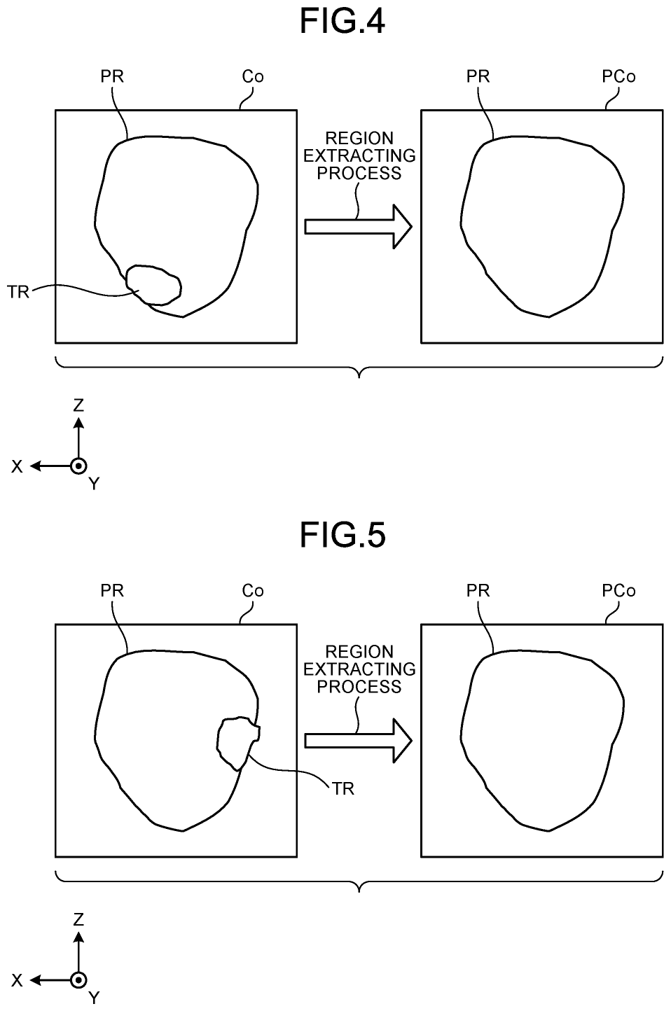

By employing the detecting function 153, the processing circuitry 15 detects a prostate region and a tumor region from the main scan images. FIGS. 4 and 5 are drawings illustrating examples of a coronal image Co related to the prostate of the patient P and another coronal image (hereinafter, “prostate image”) PCo including a prostate region PR extracted from the coronal image Co. As illustrated in FIGS. 4 and 5, in the coronal image Co prior to the execution of the region extracting process, the prostate region PR and a tumor region TR are present. In the prostate image PCo, the tumor region TR is not present, but the prostate region PR is present.

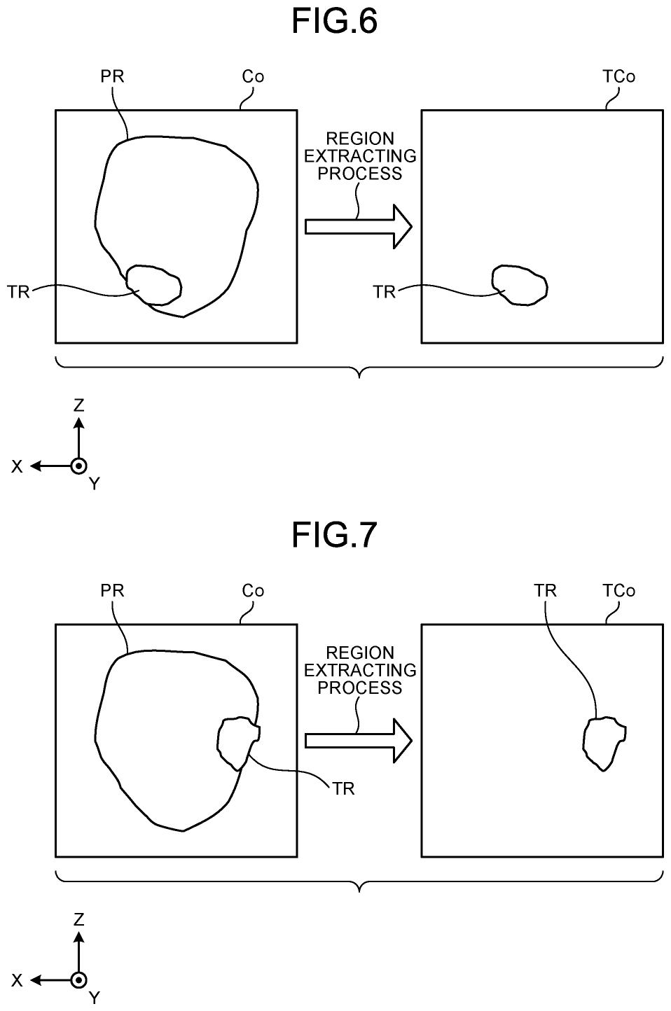

FIGS. 6 and 7 are drawings illustrating examples of a coronal image Co related to the prostate of the patient P and another coronal image (hereinafter, “tumor image”) TCo including a tumor region TR extracted from the coronal image Co. As illustrated in FIGS. 6 and 7, in the coronal image Co prior to the execution of the region extracting process, the prostate region PR and the tumor region TR are present. In the tumor image TCo, the prostate region PR is not present, but the tumor region TR is present.

Step S303:

By employing the specifying function 155, the processing circuitry 15 specifies a target boundary area of the prostate related to the tumor, on the basis of the prostate region and the tumor region. More specifically, the specifying function 155 specifies, as the target boundary area, the boundary line between the part of the tumor region TR that overlaps with the prostate region PR and the part of the tumor region TR positioned outside the prostate region PR, by using the position of the tumor region TR relative to the prostate region PR. The specifying function 155 calculates a straight line (hereinafter, “approximation straight line”) approximating the specified boundary line.

For example, the approximation straight line corresponds to the tangent line of the prostate region PR at the middle point of the boundary line, which is a straight line having an average slope on the boundary line and being in contact with the boundary line. In this situation, when the data used by the detecting function 153 is volume data, the approximation straight line may be the tangent plane of the prostate region PR at the middle point of the boundary line, which is the tangent plane having an average slope on the boundary line and being in contact with the boundary line.

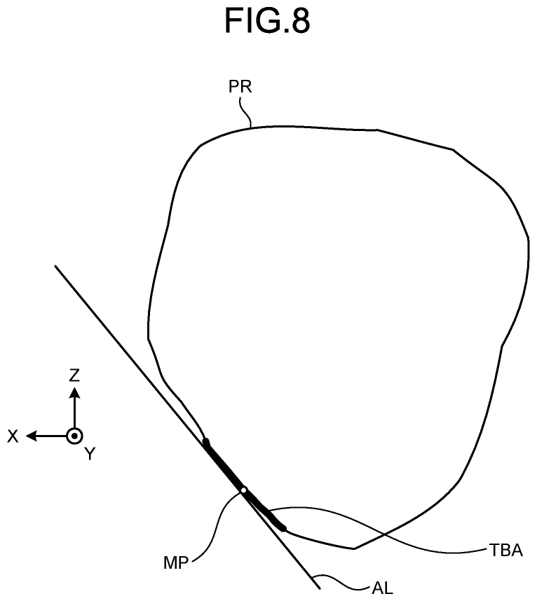

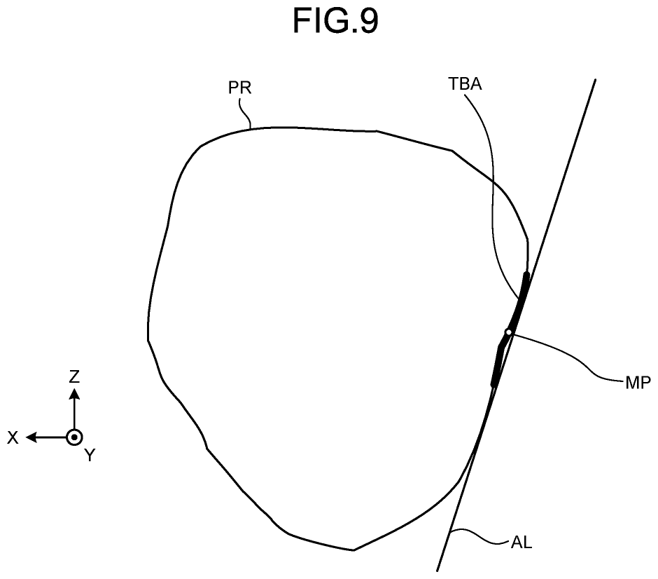

FIG. 8 is a drawing illustrating examples of a target boundary area TBA specified on the basis of the prostate region PR in FIG. 4 and the tumor region TR in FIG. 5 and an approximation straight line AL. Further, FIG. 9 is a drawing illustrating examples of a target boundary area TBA specified on the basis of the prostate region PR in FIG. 6 and the tumor region TR in FIG. 7 and an approximation straight line AL. The points “MP” in FIGS. 8 and 9 are each the middle point of the target boundary area TBA. As illustrated in FIGS. 8 and 9, the specifying function 155 specifies the target boundary area TBA by using the position of the tumor region TR relative to the prostate region PR and calculates the approximation straight line AL approximating the specified target boundary area TBA.

Step S304:

By employing the determining function 157, the processing circuitry 15 determines a perpendicular plane intersecting the boundary as a slice plane related to a diagnosis process on the tumor, by using the boundary as a reference. More specifically, by using the approximation straight line, the determining function 157 determines, as the reference plane, the slice plane that includes the middle point MP of the target boundary area TBA and the approximation straight line and is perpendicular to the coronal image Co. Subsequently, the determining function 157 determines a plurality of planes parallel to the reference plane, from the reference plane up to the two end points of the target boundary area TBA.

As a result, the determining function 157 has determined slice planes that are related to the diagnosis process on the tumor and include the reference plane and the determined plurality of planes. In this situation, when the data used by the detecting function 153 is volume data, the determining function 157 determines, as the reference plane, the slice plane that includes the middle point MP of the target boundary area TBA and is perpendicular to the tangent plane, by using the tangent plane.

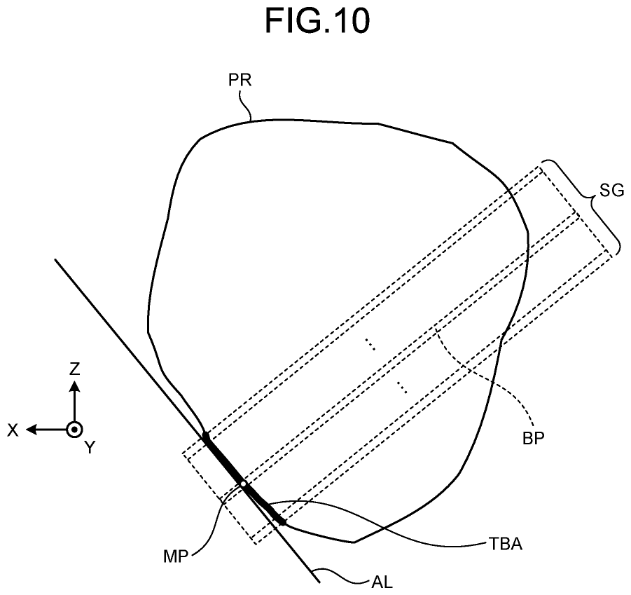

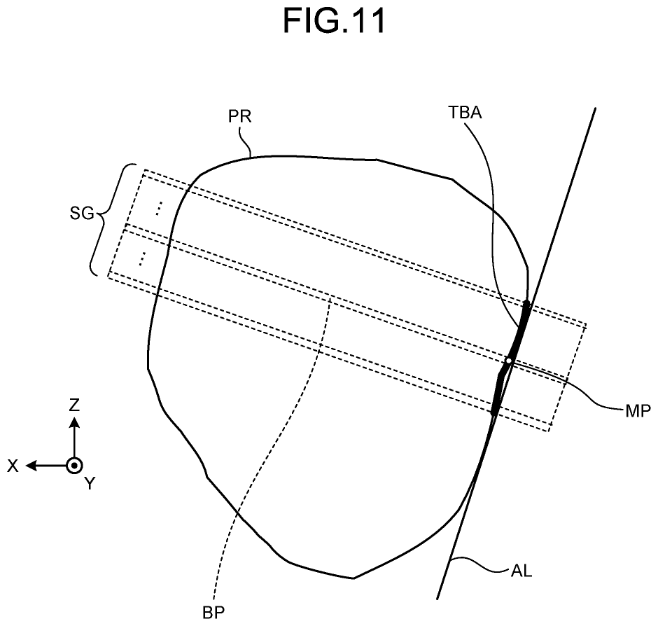

FIG. 10 is a drawing illustrating examples of a reference plane BP determined on the basis of the target boundary area TBA and the approximation straight line AL in FIG. 8 and a group of slices SG including the reference plane BP and the determined plurality of planes. FIG. 11 is a drawing illustrating examples of a reference plane BP determined on the basis of the target boundary area TBA and the approximation straight line AL in FIG. 9 and a group of slices SG including the reference plane BP and the determined plurality of planes. As illustrated in FIGS. 10 and 11, the group of slices SG including the reference plane BP correspond to desirable slice planes in an evaluation of extracapsular extension of the prostate cancer.

Step S305:

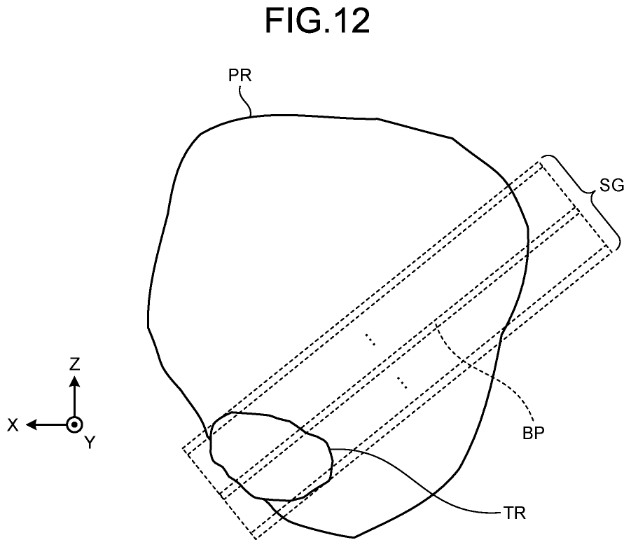

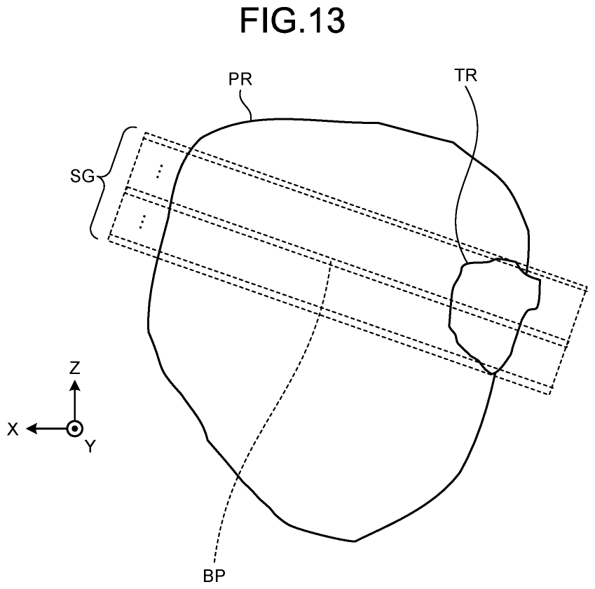

The display device 129 displays the first area on which the second area and the slice planes are superimposed. More specifically, the display device 129 displays the prostate region PR on which the tumor region TR and the group of slices SG including the determined slice plane are superimposed. FIG. 12 is a drawing illustrating an example of a superimposition image in which the prostate region PR in FIG. 4 is displayed while the tumor region TR in FIG. 6 and the group of slices SG in FIG. 10 are superimposed thereon. FIG. 13 is a drawing illustrating an example of a superimposition image in which the prostate region PR in FIG. 5 is displayed while the tumor region TR in FIG. 7 and the group of slices SG in FIG. 11 are superimposed thereon.

In this situation, also, the user is able to recognize the slice position of the reference plane BP and the range of the group of slices SG, relative to the prostate region PR and the tumor region TR. Further, the user is able to edit, via the input interface 127, the slice position of the reference plane BP and the range of the group of slices SG.

The slice determining process ends at the present step. After the present step, the imaging unit 7 performs, according to an instruction from the user via the input interface 127, an imaging process (hereinafter, “high resolution imaging process”) on the patient P to obtain a high resolution image with respect to the group of slices SG including the determined slice plane, by using the first medical image and the second medical image. By employing the reconstructing function 151, the processing circuitry 15 reconstructs a medical image (hereinafter, “high resolution image”) on the basis of MR data acquired through the high resolution imaging process. The display device 129 is configured to display the high resolution image.

Further, after the present step, according to an instruction from the user via the input interface 127, a medical image corresponding to the slice plane may be generated on the basis of volume data related to one of the first medical image and the second medical image and the determined slice plane. For example, when the user has input an instruction that medical images corresponding to the group of slices SG be generated via the input interface 127, the image generating function 159 is configured to generate the medical images corresponding to the group of slices SG, by performing an MPR process on the volume data. In this situation, the display device 129 is configured to display the medical images generated by the MPR process.

The medical image diagnosis system 1 according to the embodiment described above is configured to detect the first area corresponding to the organ of the patient P from the first medical image, to detect the second area corresponding to the tumor in the organ from one of the first medical image and the second medical image different from the first medical image, to specify the boundary of the organ on the basis of the first area and the second area, and to determine the slice plane by using the specified boundary as a reference. More specifically, the medical image diagnosis system 1 in the present example is configured to determine the slice plane so as to intersect the specified boundary. Even more specifically, the medical image diagnosis system 1 in the present example is configured to determine the slice plane so as to perpendicularly intersect the specified boundary. For example, the medical image diagnosis system 1 in the present example is configured to specify the boundary on the basis of the position of the second area relative to the first area. More specifically, the medical image diagnosis system 1 in the present example is configured to specify, as the boundary, the part of the contour of the first area that is distant from the second area by a distance equal to or shorter than the predetermined length.

With these arrangements, the medical image diagnosis system 1 in the present example is able to perform the high resolution imaging process on the patient P, with respect to the group of slices SG including the determined slice plane (the reference plane BP). Further, the medical image diagnosis system 1 in the present example is able to generate the medical images corresponding to the group of slices SG, by performing the MPR process on the volume data, with respect to the group of slices SG including the determined slice plane (the reference plane BP). Consequently, by using the medical image diagnosis system 1 in the present example, it is possible to display the medical images related to the group of slices SG including the determined slice plane (the reference plane BP).

As explained above, the medical image diagnosis system 1 in the present example is able to automatically and easily set the slice plane that is useful in diagnosis processes on the tumor. The slice plane being set is a slice plane that is useful in an evaluation of extracapsular extension of the prostate tumor, for example. Accordingly, by using the medical image diagnosis system 1 in the present example, it is possible to generate and display the medical image that is useful in the evaluation of extracapsular extension of the prostate tumor. Consequently, by using the medical image diagnosis system 1 in the present example, it is possible to automatically obtain the slice plane perpendicular to the boundary plane between the prostate tumor and the prostate, for example. It is therefore possible to improve efficiency in diagnosing extracapsular extension, by reducing burdens that may be imposed on the user at the time of setting the slice plane and making it easy to perform the extracapsular extension diagnosing processes.

When technical concepts of the embodiments of the present disclosure are realized as a medical image processing program, the medical image processing program causes a computer to realize: detecting the first area corresponding to the organ of the patient P from the first medical image; detecting the second area corresponding to the tumor related to the organ from one of the first medical image and the second medical image different from the first medical image; specifying the boundary of the organ related to the tumor on the basis of the first area and the second area; and determining the plane perpendicular to the boundary as the slice plane related to the diagnosis process on the tumor.

For example, it is also possible to realize the slice determining process by installing the medical image processing program in a computer of a modality such as the MRI apparatus 100 or of a server such as a PACS server configured to execute medical image processing processes and further loading the program into a memory. In that situation, it is also possible to distribute the program capable of causing a computer to implement the method, by storing the program in a storage medium such as a magnetic disk (e.g., a hard disk), an optical disk (e.g., a Compact Disk Read-Only Memory [CD-ROM], a Digital Versatile Disk [DVD]), or a semiconductor memory. Because the procedure and the advantageous effects of the slice determining process realized by the medical image processing program are the same as those of the embodiments of the present disclosure, explanations thereof will be omitted.

Further, technical concepts of the embodiments of the present disclosure may be applied to a modality different from the MRI apparatus 100 such as an X-ray computed tomography (hereinafter, “CT”) apparatus or an ultrasound diagnosis apparatus. In this situation, for example, the imaging unit 7 corresponds to a unit configured to obtain raw data in the X-ray CT apparatus or the ultrasound diagnosis apparatus. In addition, the reconstructing function 151 corresponds to a unit configured to generate a CT image or an ultrasound image on the basis of the raw data. Because the procedure and the advantageous effects of the slice determining process performed by the X-ray CT apparatus or the ultrasound diagnosis apparatus are the same as those of the embodiments of the present disclosure, explanations thereof will be omitted.

According to at least one aspect of the embodiments and the like described above, it is possible to easily set the slice plane that is useful in diagnosis processes on the tumor. As a result, according to at least one aspect of the embodiments of the present disclosure, it is possible to improve efficiency in diagnosing extracapsular extension, by reducing burdens that may be imposed on the user at the time of setting the slice plane and making it easy to perform the extracapsular extension diagnosing processes.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis system comprising processing circuitry configured:
to detect a first area corresponding to an organ of an examined subject from a first medical image and to detect a second area corresponding to a tumor in the organ from one of the first medical image and a second medical image different from the first medical image;
to specify a boundary of the organ on a basis of the first area and the second area; and
to determine a slice plane by using the boundary as a reference.

2. The medical image diagnosis system according to claim 1, wherein the processing circuitry specifies the boundary, on a basis of a position of the second area relative to the first area.

3. The medical image diagnosis system according to claim 1, wherein, as the boundary, the processing circuitry specifies a part of a contour of the first area that is distant from the second area by a distance equal to or shorter than a predetermined length.

4. The medical image diagnosis system according to claim 1, wherein the processing circuitry determines the slice plane so as to intersect the boundary.

5. The medical image diagnosis system according to claim 1, wherein the processing circuitry determines the slice plane so as to perpendicularly intersect the boundary.

6. The medical image diagnosis system according to claim 1, further comprising: a display device configured to display the first area on which the second area and the slice plane are superimposed.

7. The medical image diagnosis system according to claim 1, further comprising:
an imaging apparatus configured to perform, on the examined subject, an imaging process with respect to the slice plane so as to obtain a high resolution image by using the first medical image and the second medical image; and
a display device configured to display a medical image generated by the imaging process and corresponding to the slice plane.

8. The medical image diagnosis system according to claim 1, wherein
the processing circuitry generates a medical image corresponding to the slice plane, on a basis of volume data related to one of the first and the second medical images and the slice plane, and
the medical image diagnosis system further comprises a display device configured to display the medical image corresponding to the slice plane.

9. The medical image diagnosis system according to claim 1, wherein the organ is a prostate.

10. The medical image diagnosis system according to claim 1, wherein the medical image diagnosis system is a magnetic resonance imaging system.

11. A non-transitory computer-readable storage medium storing therein a medical image processing program that causes a computer to realize:
detecting a first area corresponding to an organ of an examined subject from a first medical image;
detecting a second area corresponding to a tumor related to the organ from one of the first medical image and a second medical image different from the first medical image;
specifying a boundary of the organ related to the tumor on a basis of the first area and the second area; and
determining a plane perpendicular to the boundary as a slice plane.
